(19) United States
(12) Patent Application Publication
(10) Pub. No.: US 2025/0035601 A1
(43) Pub. Date: Jan. 30, 2025

(54) CHEMICAL ANALYSIS PROCESS IN GAS PHASE

(71) Applicant: Hemera, Meylan (FR)

(72) Inventor: Rachid Mouflih, Meylan (FR)

(21) Appl. No.: 18/782,488

(22) Filed: Jul. 24, 2024

(30) Foreign Application Priority Data
Jul. 27, 2023 (FR) 2308127

(51) Int. Cl. G01N 33/00; G01N 21/31

(52) U.S. Cl. CPC G01N 33/0042; G01N 21/3103; G01N 33/0024

(57) ABSTRACT

A method for chemical analysis of a gaseous sample comprising a first species, the first species exhibiting a spectroscopic first activity in a predetermined wavelength range, comprises:
converting the first species into a second species by reaction with a conversion reagent in a conversion cell, the second species exhibiting a spectroscopic second activity greater than the first activity;
acquiring an absorption spectrum by spectroscopic measurement of the gaseous sample in a measuring cell fluidically connected to the conversion vial after the converting of the first species into the second species; and
mathematically processing the absorption spectrum to deduce a concentration of the first species in the gaseous sample.

### PRIORITY CLAIM

This application claims the benefit of the filing date of French Patent Application Serial No. FR2308127, filed Jul. 27, 2023, for “GAS-PHASE CHEMICAL ANALYSIS METHOD.”

### TECHNICAL FIELD

The present disclosure relates to the field of chemical analysis, and more particularly to gas-phase chemical analysis.

In particular, the present disclosure relates to a method for the chemical analysis of a gaseous sample comprising at least one contaminant species with a view to determining the concentration of the at least one contaminant species in the sample.

### BACKGROUND

Detecting and quantifying contaminant species in industrial gases is essential to guarantee their degree of purity.

In these regards, it is possible to use spectroscopic techniques that are not only stable over time, but also have the advantage of being relatively easy to implement. Nevertheless, there are situations where contaminant species are difficult to detect and/or identify by spectroscopy. This is particularly true of H₂S. This compound, with its low spectroscopic activity, is barely detectable when its concentration in a gas is less than 1 ppm.

Thus, one aim of the present disclosure is to provide a method for the chemical analysis of a gaseous sample comprising a contaminant species, with greater sensitivity than methods known to the skilled person.

### BRIEF SUMMARY

The present disclosure relates to a method for chemical analysis of a gaseous sample comprising a first species, the first species exhibiting a spectroscopic activity, called first activity, in a predetermined wavelength range, the method of chemical analysis comprising:
a) a step for conversion, in a conversion cell, of the first species into a second species by reaction with a reagent, called conversion reagent, the second species exhibiting a spectroscopic activity, called second activity, greater than the first activity;
b) a step for acquiring an absorption spectrum, by spectroscopic measurement, of the gaseous sample after step a) has been carried out in a measuring cell fluidically connected to the conversion cell;
c) a step for mathematical processing of the spectrum to deduce the concentration of the first species in the gaseous sample.

According to one embodiment, the second spectroscopic activity is at least ten times greater than the first spectroscopic activity.

According to one embodiment, the second species exhibits a substantially periodic absorption spectrum in the predetermined wavelength range.

According to one embodiment, the first species comprises H₂S, while the second species comprises SO₂.

According to one embodiment, the conversion reagent comprises I₂O₅.

According to one embodiment, the conversion reagent is in one of the forms chosen from: powder, pellet, flakes, granules, beads.

According to one embodiment, step b) comprises measuring by way of a spectrograph the absorption spectrum by the gaseous sample of light radiation emitted by a light source.

### BRIEF DESCRIPTION OF THE DRAWINGS

Other features and advantages of the present disclosure will emerge from the following detailed description of the present disclosure with reference to the appended figure, wherein:

FIG. 1 is a pictorial of an absorption spectrum of H₂S between 185 nm and 225 nm, with the vertical axis representing the absorption level (in arbitrary units) and the vertical axis representing the wavelength in nm;

FIG. 2 is a pictorial of an absorption spectrum of SO₂ between 185 nm and 225 nm, with the vertical axis representing the absorption level (in arbitrary units) and the vertical axis representing the wavelength in nm;

FIG. 3 is a schematic depiction of an analysis device capable of implementing the analysis method according to the present disclosure.

### DETAILED DESCRIPTION

The present disclosure relates to a method for the chemical analysis of a gaseous sample comprising a carrier gas wherein a first species is diluted. In particular, the first species, according to the terms of the present disclosure, has a spectroscopic activity, known as the first activity, in a predetermined range of wavelengths.

Spectroscopic activity according to the terms of the present disclosure can be defined as the level of spectroscopic absorption in the predetermined wavelength range.

Thus, the analysis method according to the present disclosure comprises a step a) for conversion, in a conversion cell, of the first species into a second species by reaction with a reagent, called conversion reagent, the second species exhibiting a spectroscopic activity, called second activity, greater than the first activity;

The method also comprises a step b) for acquiring an absorption spectrum, by spectroscopic measurement, of the gaseous sample after step a) has been carried out, in a measuring cell fluidically connected to the conversion cell;

Step b) is followed by a step c) for mathematical processing of the spectrum to deduce the concentration of the first species in the gaseous sample.

Under the terms of the present disclosure, the first species exhibits spectroscopic activity, called first activity, in the predetermined wavelength range, while the second species exhibits spectroscopic activity, called second activity, in the predetermined wavelength range. The second spectroscopic activity can, for example, be at least 2 times, advantageously 10 times, greater than the first spectroscopic activity.

In other words, and according to the present disclosure, in the predetermined wavelength range, the maximum absorption level (i.e., spectroscopic activity) of the second species is greater (advantageously at least twice, advantageously 10 times, greater) than the maximum absorption level of the first species.

In addition, and advantageously, the second species can exhibit a specific signature in the predetermined wavelength range. In particular, the second species can exhibit a periodic or pseudoperiodic absorption spectrum enabling unambiguous identification of the second species. A periodic or pseudoperiodic absorption spectrum takes the form of a comb.

By way of example, and without being limiting, it will be considered in the following that the first species is H₂S while the second species is SO₂. The sample gas thus comprises a carrier gas wherein H₂S is diluted. The latter can be converted to SO₂ using a conversion reagent, such as I₂O₅.

The conversion reaction of H₂S to SO₂ by I₂O₅ follows the following reaction:

5H₂S+3I₂O₅→3I₂+5SO₂+5H₂O

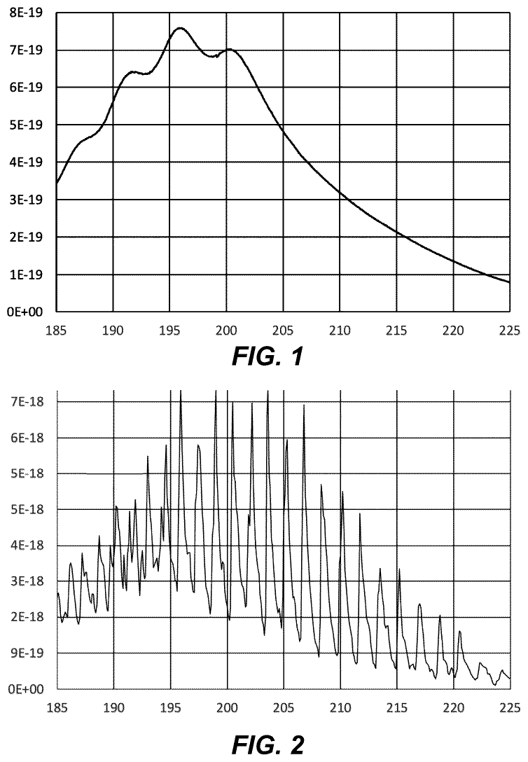

By way of example, FIG. 1 shows a pictorial of the absorption spectrum of H₂S (taken as the first species) in the wavelength range 185 nm-225 nm, while FIG. 2 shows the absorption spectrum of SO₂ (taken as the second species) in the same wavelength range.

These two pictorials show that the absorption level of SO₂ in this wavelength range is higher than the absorption level of H₂S.

In other words, the detection threshold of H₂S by spectroscopic absorption is much better than that of SO₂. By converting H₂S into SO₂, the presence of H₂S can be detected at lower concentrations than with techniques known from the state of the art based on direct absorption of the H₂S compound.

This last aspect improves the sensitivity of assay and/or analysis of a chemical compound diluted in a carrier gas.

Furthermore, the periodic nature of the absorption spectrum shown in FIG. 2 is characteristic of the presence of SO₂. In particular, the measured period indicates the presence of SO₂, while the amplitude of the oscillations is associated with the concentration of SO₂ in the carrier gas.

Mathematical processing, in particular, involving a Fourier transform, can be used to determine the SO₂ concentration in the carrier gas.

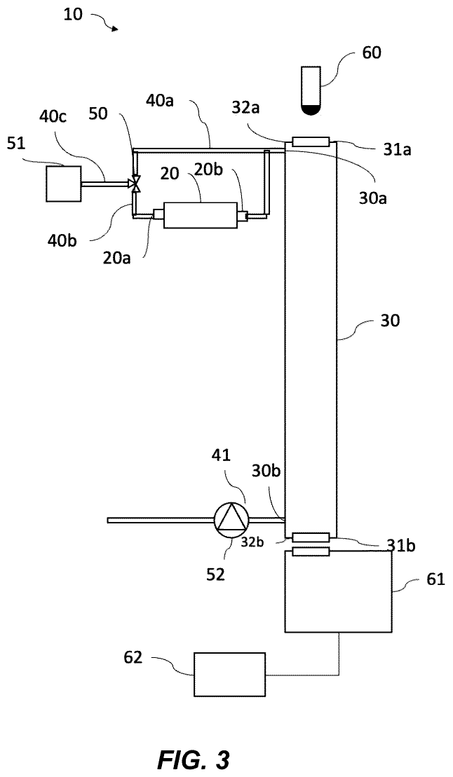

By way of example, the chemical analysis method according to the present disclosure can be implemented by the chemical analysis device 10 shown in FIG. 3.

The chemical analysis device 10 comprises, in particular, a conversion cell 20 (e.g., a vial) wherein a conversion reagent is arranged, configured to convert the first species of the gaseous sample into the second species.

The conversion cell 20 is generally elongated, and may, for example, be cylindrical. The conversion cell 20 comprises a first inlet 20a at one end and a first outlet 20b at the other end.

The conversion cell 20 comprises, in its volume, a reagent, called conversion reagent, configured to chemically convert the first species likely to circulate in gaseous form in the cell into the second species.

The conversion reagent can, in particular, comprise I₂O₅, for example, in powder or even crystal form. Thus, to prevent I₂O₅ from being carried along by the carrier gas as it flows, membranes, such as PTFE membranes, may be considered.

The chemical analysis device 10 also comprises spectroscopic means configured to measure an absorption spectrum of the gaseous sample after conversion of dichlorine to chlorine dioxide.

More particularly, the spectroscopic means cooperate with a measuring cell 30 (e.g., a vial) to measure the absorption spectrum when the gaseous sample is in the measuring cell.

More particularly, the generally elongated measuring cell 30 comprises two ends 31a and 31b (opposite one another), each provided with a window 32a and 32b transparent to the wavelengths included in the analysis range. These two windows 32a and 32b are parallel to one another.

The conversion cell 20 can comprise a main body, tubular in shape and hermetically sealed at its ends by PTFE membranes that allow gases to pass through and retain the conversion reagent.

Advantageously, the main body can be transparent and comprise, for example, PMMA or glass.

The spectroscopic means comprise a spectrograph 61 and a light source 60 configured to emit light radiation covering the analysis range.

In particular, the light source 60 is arranged opposite the window 32a to enable illumination of a gaseous sample likely to be present in the measuring cell 30.

The spectrograph 61 is positioned opposite the window 32b and is arranged to measure the absorption of light radiation by the gaseous sample in the analysis range. It is understood, but not necessary to specify, that a spectrograph measures an absorption spectrum as a function of wavelength. The spectrograph 61 is advantageously connected to a computer 62 configured to process and/or analyze the spectra collected by the spectrograph 61.

“Computer” is understood to mean an electronic device configured to perform automatic processing of a spectrum collected by the spectrograph. The computer 62 can comprise a memory, a processor, a motherboard, a display and a control interface.

The measuring cell 30 is also fluidically connected to the conversion cell 20. In particular, a fluidic connection means connects the first outlet 20b to an inlet, called second inlet 30a, located proximate to the end 31a of the measuring cell 30. In particular, the fluidic connection means may comprise a fluidic connection tube, called first tube 40a. The fluidic connection means can also be provided with a three-way valve (or solenoid valve) 50. Thus, the fluidic connection means may comprise a tube 40b connecting the first outlet 20b to the three-way valve 50, the tube 40a connecting the three-way valve 50 to the second inlet 30a, and finally a tube 40c connecting a carrier gas source 51 to the three-way valve 50.

The measuring cell 30 also comprises an outlet, called second outlet 30b, located proximate to the end 31b. This second outlet 30b can notably be connected to a pump 52 by way of a fluidic connection tube, called second tube 41. In particular, this latter arrangement can be used to purge and/or transfer the gaseous sample from the measuring cell.

The remainder of this description discloses a method for the chemical analysis of a gaseous sample comprising a carrier gas wherein H₂S is diluted. In particular, the carrier gas may comprise dinitrogen, CH4 or H₂, without, however, limiting the present disclosure to these gases alone.

In a preferred embodiment, the conversion reagent comprises I₂O₅, in powder or single-crystal form.

The reaction between H₂S and I₂O₅ is written as follows:

5H₂S+3I₂O₅→3I₂+5SO₂+5H₂O

The chemical analysis method also comprises a step b) for acquiring an absorption spectrum of the gaseous sample after step a) has been carried out in the measuring cell 30.

In these regards, the gas sample is transferred (step al)) from the conversion cell 20 to the measuring cell 30 via the fluidic connection means. In particular, this transfer may involve positioning the three-way valve 50 so as to allow the gas sample to flow from the first outlet 20b to the second inlet 30a.

Acquisition of the absorption spectrum involves illumination of the gas sample present in the measuring cell 30 by the light source 60 and collection of the light signal, after absorption by the gas sample, by the spectrograph 61.

In the 185 nm-225 nm wavelength range, SO₂ has a specific “comb” signature, as shown in FIG. 2.

The present disclosure also comprises a step c) of mathematically processing the absorption spectrum to deduce the concentration of SO₂ in the gaseous sample.

Knowing the SO₂ concentration in the gaseous sample gives access to the initial H₂S concentration in the gaseous sample.

Mathematical processing step c) can comprise a Fourier transform calculation of the absorption spectrum. In particular, the Fourier transform of the spectrum shown in FIG. 2 is a bell-shaped curve whose area is representative of the concentration of SO₂ present in the gaseous sample. The skilled person will recognize that calibration curves and/or abacuses can be used to interpret the measurements done.

Advantageously, mathematical processing step c) can be carried out by way of the computer 62.

Thus, the present disclosure proposes to determine the concentration of diluted H₂S in a carrier gas by converting it into SO₂. The latter has a higher absorption level than H₂S, between 185 nm and 225 nm, and can therefore be used to measure H₂S concentrations in a sample below the thresholds usually accepted when using analytical techniques known to the skilled person.

The chemical analysis method can comprise a background measurement step a0), preceding step a), which comprises measuring the absorption spectrum of a gaseous sample, referred to as background, formed solely of the carrier gas. This step a0) notably involves acquiring an absorption spectrum of the (dichlorine-free) carrier gas present in the measuring cell in the same way as in step b).

In particular, the carrier gas may have been taken from the carrier gas source 51.

Of course, the present disclosure is not limited to the described embodiments and variant embodiments may be envisaged without departing from the scope of the invention as defined by the claims.

1. A method for chemical analysis of a gaseous sample comprising a first species, the first species exhibiting a first spectroscopic activity in a predetermined wavelength range, the method comprising:
converting the first species into a second species by reaction with a conversion reagent in a conversion cell, the second species exhibiting a second spectroscopic activity greater than the first spectroscopic activity;
acquiring an absorption spectrum by spectroscopic measurement of the gaseous sample in a measuring cell fluidically connected to the conversion vial after converting the first species into the second species; and
mathematically processing the absorption spectrum to deduce a concentration of the first species in the gaseous sample.

2. The method of claim 1, wherein the second spectroscopic activity is at least twice the first spectroscopic activity.

3. The method of claim 2, wherein the second species exhibits a substantially periodic absorption spectrum in the predetermined wavelength range.

4. The method of claim 3, wherein the first species comprises H₂S and the second species comprises SO₂.

5. The method of claim 4, wherein the conversion reagent comprises I₂O₅.

6. The method of claim 5, wherein the conversion reagent is in a form chosen from among: powder, pellets, flakes, granules, or beads.

7. The method of claim 6, wherein acquiring the absorption spectrum by spectroscopic measurement comprises using a spectrograph to measure the absorption spectrum by the gaseous sample of light radiation emitted by a light source.

8. The method of claim 2, wherein the second spectroscopic activity is at ten times the first spectroscopic activity.

9. The method of claim 1, wherein the second species exhibits a substantially periodic absorption spectrum in the predetermined wavelength range.

10. The method of claim 1, wherein the first species comprises H₂S and the second species comprises SO₂.

11. The method of claim 10, wherein the conversion reagent comprises I₂O₅.

12. The method of claim 11, wherein the conversion reagent is in a form chosen from among: powder, pellets, flakes, granules, or beads.

13. The method of claim 1, wherein acquiring the absorption spectrum by spectroscopic measurement comprises using a spectrograph to measure the absorption spectrum by the gaseous sample of light radiation emitted by a light source.
